(19) United States
(12) Patent Application Publication
(10) Pub. No.: US 2017/0340443 A1
(43) Pub. Date: Nov. 30, 2017

(54) POSTERIOR MITRAL VALVE LEAFLET APPROXIMATION

(71) Applicant: Edwards Lifesciences Corporation, Irvine, CA (US)

(72) Inventors: Grant Matthew Stearns, Costa Mesa, CA (US); David M. Taylor, Lake Forest, CA (US)

(73) Assignee: Edwards Lifesciences Corporation, Irvine, CA (US)

(21) Appl. No.: 15/599,219

(22) Filed: May 18, 2017

Related U.S. Application Data
(60) Provisional application No. 62/340,786, filed on May 24, 2016.

(51) Int. Cl. A61F 2/24

(52) U.S. Cl. CPC A61F 2/2454; A61F 2/2466; A61F 2220/0016

(57) ABSTRACT

The present disclosure provides embodiments of a method for improving coaptation of the anterior and posterior mitral valve leaflets by applying a remodeling force to the posterior leaflet. In particular embodiments, a tension member is secured at a location on or proximate to the posterior leaflet. Tension can be applied to the tension member in a direction superiorly and anteriorly toward the interatrial septum. The tension member can be secured at a location proximate the septum to maintain the tension. The tension provides the remodeling force, pulling the posterior leaflet superiorly and anteriorly to improve coaptation with the anterior leaflet.

### CROSS REFERENCE TO RELATED APPLICATION

This application claims the benefit of U.S. Provisional Patent Application No. 62/340,786, filed May 24, 2016, which is incorporated herein by reference.

### FIELD

This disclosure pertains generally to methods for preventing or reducing regurgitation through heart valves, and delivery systems and implantable devices useable in such methods.

### BACKGROUND

The native heart valves (i.e., the aortic, pulmonary, tricuspid, and mitral valves) serve critical functions in assuring the forward flow of an adequate supply of blood through the cardiovascular system. These heart valves can be rendered less effective by congenital malformations, inflammatory processes, infectious conditions, or disease. Such damage to the valves can result in serious cardiovascular compromise or death. For many years, the definitive treatment for such disorders was the surgical repair or replacement of the valve during open-heart surgery. However, such surgeries are highly invasive and are prone to many complications. Therefore, elderly and frail patients with defective heart valves often went untreated. More recently, transvascular techniques have been developed for introducing and implanting prosthetic devices in a manner that is much less invasive than open-heart surgery. Such transvascular techniques have increased in popularity due to their high success rates.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downwardly from the annulus into the left ventricle. The mitral valve annulus can form a D-shaped, oval, or otherwise out-of-round cross-sectional shape having major and minor axes. The anterior leaflet can be larger than the posterior leaflet, forming a generally C-shaped boundary between the abutting free edges of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates, the oxygenated blood that is collected in the left atrium flows into the left ventricle. When the muscles of the left atrium relax and the muscles of the left ventricle contract, the increased blood pressure in the left ventricle urges the two leaflets of the mitral valve together, thereby closing the one-way mitral valve so that blood cannot flow back into the left atrium and is, instead, expelled out of the left ventricle through the aortic valve. To prevent the two leaflets from prolapse under pressure and folding back through the mitral valve annulus towards the left atrium, a plurality of fibrous cords called chordae tendineae tether the leaflets to papillary muscles in the left ventricle.

Mitral regurgitation occurs when the native mitral valve fails to close properly and blood flows into the left atrium from the left ventricle during the systole phase of the cardiac cycle. Mitral regurgitation is the most common form of valvular heart disease. Mitral regurgitation has different causes, such as leaflet prolapse, dysfunctional papillary muscles, and/or stretching of the mitral valve annulus resulting from dilation of the left ventricle. Mitral regurgitation at a central portion of the leaflets can be referred to as central jet mitral regurgitation, and mitral regurgitation nearer to one commissure (i.e., location where the leaflets meet) of the leaflets can be referred to as eccentric jet mitral regurgitation.

Some prior techniques for treating mitral regurgitation include stitching edge portions of the native mitral valve leaflets directly to one another (known as an Alfieri stitch). Other prior techniques include the implantation of a fixation member that mimics an Alfieri stitch by fixing edge portions of the native leaflets to one another. One commercially available fixation device is the Mitraclip®, available from Evalve, Inc. A substantial number of patients treated with an Alfieri stitch or a fixation member have experienced poor clinical outcome, that is, significant residual mitral regurgitation. In some cases, residual mitral regurgitation can be treated by implanting one or more additional fixation members or additional stitches. However, additional fixation members or stitches can increase the pressure gradient across the mitral valve to an unacceptable level.

A structural feature of the heart that can be associated with mitral valve regurgitation is an increase in the septal-lateral dimension of the mitral annulus. One technique that has been applied to address this feature is septal-lateral annular cinching. In particular, a percutaneous septal sinus shortening system (PS³ System™, Ample Medical, Foster City, Calif.) was used in preclinical studies to attempt to reduce the septal-lateral dimension of the mitral annulus. In this procedure, a T-bar was inserted into the left atrium using a catheter placed in the great cardiac vein and a catheter placed in the left atrium using a transseptal puncture. The T-bar was inserted into the coronary sinus. A wire connected to the T-bar was secured to an interatrial septum anchor and tensioned to cause septal-lateral shortening. The use of multiple delivery systems can make this system complex to implement.

### SUMMARY

The present disclosure relates to embodiments that promote coaptation of the leaflets of a heart valve by applying tension with a tension member secured at a location on or proximate a leaflet. Particular described embodiments relate to treating a mitral valve. However, it should be understood that any of the disclosed embodiments can be used to treat the other valves of the heart (e.g., the aortic, pulmonary, and tricuspid valves). When used to treat a mitral valve, in particular implementations, the tension member can be secured on or proximate the base of the posterior mitral valve leaflet, the posterior annulus, or a location superior to the posterior annulus and inferior to the coronary sinus.

In one representative embodiment, the tension member is deployed proximate the posterior mitral valve leaflet, such as by being deployed from a catheter. For example, a deployment catheter can be advanced through the interatrial septum of the heart to a location proximate the posterior mitral valve leaflet. The tension member can be secured at the location and can extend from the location, such as through the posterior mitral valve leaflet, and through the interatrial septum. The tension member can comprise, for example, an elongated, flexible piece of material, such as a suture, string, coil, cable, cord, wire, or similar material. The tension member can be secured proximate the interatrial septum to apply a desired tension to the tension member, and in turn to the posterior mitral valve leaflet. In some embodiments, the tension member applies an anteriorly and superiorly directed force to the posterior mitral valve leaflet, pulling the leaflet and the chordae tendineae closer to the interatrial septum and the anterior mitral valve leaflet. The force can improve coaptation of the posterior mitral valve leaflet with the anterior mitral valve leaflet.

In some implementations, the tension member can be secured to the posterior mitral valve leaflet, such as with a loop of the tension member. For example, the posterior mitral valve leaflet can be penetrated and the tension member can be passed through the tissue of the posterior mitral valve leaflet. A portion of the tension member can be secured beneath the posterior mitral valve leaflet, such as to form one or more loops through the leaflet.

In other implementations, the tension member can be secured to the leaflet by deploying an anchor device coupled to the tension member adjacent the inferior surface of the posterior mitral valve leaflet. In particular examples, the anchor device can include one or more gripping elements, such as barbs or spikes, to help secure the anchor device to the leaflet tissue. In further examples, the anchor device can have a delivery configuration to facilitate advancement of the anchor device through a catheter and into the heart, and a deployed configuration to facilitate securing the anchor device to, or otherwise engaging the anchor device with, the tissue of the posterior mitral valve leaflet to apply a remodeling force. In a specific example, the anchor device can have a bent or disjoint configuration during delivery and a straight or aligned configuration when deployed inside a patient's heart.

In further implementations, the tension member can be secured on or proximate to the leaflet with an anchor member comprising one or more gripping elements, such as barbs. The gripping elements can extend axially in a distal direction while the anchor member is inside a catheter. When advanced from the catheter, the gripping elements can bend radially outwardly and proximally, such as to form a hook-like shape. The gripping elements can secure the anchor member in heart or leaflet tissue on or proximate to the leaflet.

In a representative embodiment, the tension member can be secured proximate the interatrial septum using a closure member. The closure member can be implanted in the interatrial septum and configured to close an opening in the interatrial septum, such as an opening through which the tension member extends. The tension member can extend through the closure member. The tension member can be secured relative to the closure member using a fastener adjacent the closure member, such as a suture clip or locking or retaining device. In particular implementations, the fastener can be advanced over the tension member until it is proximate the closure member. When a desired degree of tension has been applied to the tension member, the fastener can be secured to the tension member so that the tension is maintained, such as when an excess portion of the tension member is severed. In a particular example, the fastener is coupled to the tension member proximate a right atrial surface of the closure member.

In certain embodiments, the tension member can be deployed from a catheter. A free end of the tension member can be retrieved with a snare member of a snare catheter and retracted through the interatrial septum. The free end of the tension member can be withdrawn into the snare catheter. In some cases, two ends of the tension member can be retrieved and withdrawn into the snare catheter. The remodeling force can then be applied to the free end of the tension member, and the tension member secured with a fastener and/or closure device as described above.

In another representative embodiment, the present disclosure provides an assembly useable to promote coaptation of the mitral valve leaflets according to a method described above. The delivery assembly can include one or more of various components useable in the method, including the tension member, anchor device, snare member, closure member, or fastener. The delivery assembly can include one or more catheters into which the components are loaded. In particular implementations, the assembly includes a delivery catheter and one or both of a deployment catheter and a snare catheter which may be disposed within the delivery catheter. In a particular example, the deployment catheter can be used to deploy the tension member, and, optionally, the anchor device within the heart. The snare catheter can be used to retrieve a free end of the tension member, apply a tensioning force to the tension member, and secure a fastener to the tension member. The snare catheter can also be configured to deploy the closure member proximate the interatrial septum. Once loaded into the catheter (or catheters) the various components can be advanced in turn in order to carry out a disclosed method.

The foregoing and other objects, features, and advantages of the invention will become more apparent from the following detailed description, which proceeds with reference to the accompanying figures.

### BRIEF DESCRIPTION OF THE DRAWINGS

FIGS. 1-6 are cross sections of a heart showing the deployment of a tension member and an anchor device transseptally to the posterior mitral valve leaflet according to an embodiment of the present disclosure.

FIG. 7A is a cross section of a heart showing an anchor device disposed beneath the posterior mitral valve leaflet and coupled using a tension member to a closure member implanted in the interatrial septum of the heart.

FIG. 7B is a top plan view of the mitral valve showing an anchor member disposed beneath the posterior mitral valve leaflet and a tension member passing through the posterior leaflet.

FIGS. 8A and 8B show an exemplary embodiment of an anchor device coupled to a tension member with the anchor device shown in a delivery configuration and deployed configuration, respectively.

FIGS. 9A and 9B show an exemplary embodiment of an anchor device coupled to a tension member with the anchor device shown in a deployed configuration and a delivery configuration, respectively.

FIGS. 10-15 are cross sections of a heart showing the deployment of a tension member transseptally to the posterior mitral valve leaflet, and securing the tension member to the leaflet, according to an embodiment of the present disclosure.

FIG. 16A is an enlarged view of the posterior mitral valve leaflet showing a tension member passing through the posterior leaflet at multiple locations along the width of the leaflet.

FIG. 16B is a top plan view of the mitral valve showing a tension member passing through the posterior leaflet at multiple locations along the length of the leaflet.

FIG. 17 is a side view of a delivery catheter for use in deploying a tension member proximate the posterior mitral valve leaflet, according to one embodiment.

FIG. 18 is a side view of an embodiment of a laser cut tube that can be used in the steerable section of the delivery catheter shown in FIG. 17.

FIG. 19 is a cross sectional view of the delivery catheter of FIG. 17 taken along line 19-19.

FIG. 20 is an enlarged side view of the shaft of the delivery catheter of FIG. 17.

FIG. 21 is a perspective view of an embodiment of a deployment catheter that can be used with the delivery catheter of FIG. 17 to deploy a tension member proximate the posterior mitral valve leaflet.

FIG. 22 is a perspective view of an embodiment of a needle wire for puncturing native leaflet tissue.

FIGS. 23 and 24 are perspective views of two different embodiments of a snare catheter that can be used with the delivery catheter of FIG. 17 when deploying a tension member proximate the posterior mitral valve leaflet.

FIG. 25 is a side view of an embodiment of a tension member-feeding device that can be used to advance a tension member through a catheter.

FIG. 26 is a cross section of a heart showing anchoring locations for a tension member according to an embodiment of the present disclosure.

FIGS. 27-28 are cross sections of a heart showing the deployment of a tension member and an anchor device transseptally to the posterior mitral valve leaflet according to an embodiment of the present disclosure.

### DETAILED DESCRIPTION

For purposes of this description, certain aspects, advantages, and novel features of the embodiments of this disclosure are described herein. The described methods, systems, and apparatus should not be construed as limiting in any way. Instead, the present disclosure is directed toward all novel and nonobvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect, embodiment or example of the invention are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The present disclosure is not restricted to the details of any foregoing embodiments. The present disclosure extends to any novel one, or any novel combination, of the features disclosed in this specification (including any accompanying claims, abstract, and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

As used herein, the terms “a,” “an,” and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A,” “B,” “C,” “A and B,” “A and C,” “B and C,” or “A, B, and C.”

As used herein, the term “coupled” generally means physically coupled or linked and does not exclude the presence of intermediate elements between the coupled items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device toward the user, while distal motion of the device is motion of the device away from the user. The terms “longitudinal” and “axial” refer to an axis extending in the proximal and distal directions, unless otherwise expressly defined.

The present disclosure can provide methods for reducing the distance between the interatrial septum and the posterior mitral valve leaflet of the heart, as well as systems and devices for carrying out such methods. Reducing this distance can promote coaptation of the mitral valve leaflets, thus reducing mitral valve regurgitation. The methods can generally include using a delivery system to create a transseptal puncture. A tension member can be passed through the transseptal puncture and secured to the posterior mitral valve leaflet. A septal closure member can be placed proximate the transseptal puncture. The tension member can be coupled to the septal closure device, and can be tensioned to provide a desired degree of reduction in the distance between the interatrial septum and the posterior mitral valve leaflet. The tension member can be secured to maintain the tension using a fastener.

In one implementation, the tension member can be secured to the posterior leaflet using an anchoring device, such as a bar or rod. In another implementation, the tension member may be looped through the superior and inferior surfaces of the posterior mitral valve leaflet one or more times. The tension member can be placed under tension, thus pulling the posterior mitral valve leaflet anteriorly and superiorly, towards the interatrial septum, which can promote coaptation between the mitral valve leaflets.

The disclosed methods, systems, and devices can provide various advantages. For example, the disclosed methods can be carried out with a single delivery system. Compared with prior methods involving multiple delivery systems, the disclosed methods can be less procedurally complex. For example, compared with other septal-lateral annular cinching techniques, the disclosed methods can avoid catheterization of the coronary sinus. As another example, the disclosed technique can avoid complex maneuvers requiring the coordination of multiple, non-coaxial catheters.

In addition to potential procedural simplicity, the disclosed methods can provide for improved coaptation between the mitral valve leaflets. For example, other septal-lateral annular cinching techniques attempt to promote coaptation by pulling on the coronary sinus. The disclosed technique can directly pull the posterior mitral valve leaflet towards the interatrial septum, which can require less tension and displacement of the left atrium than techniques that pull the coronary sinus.

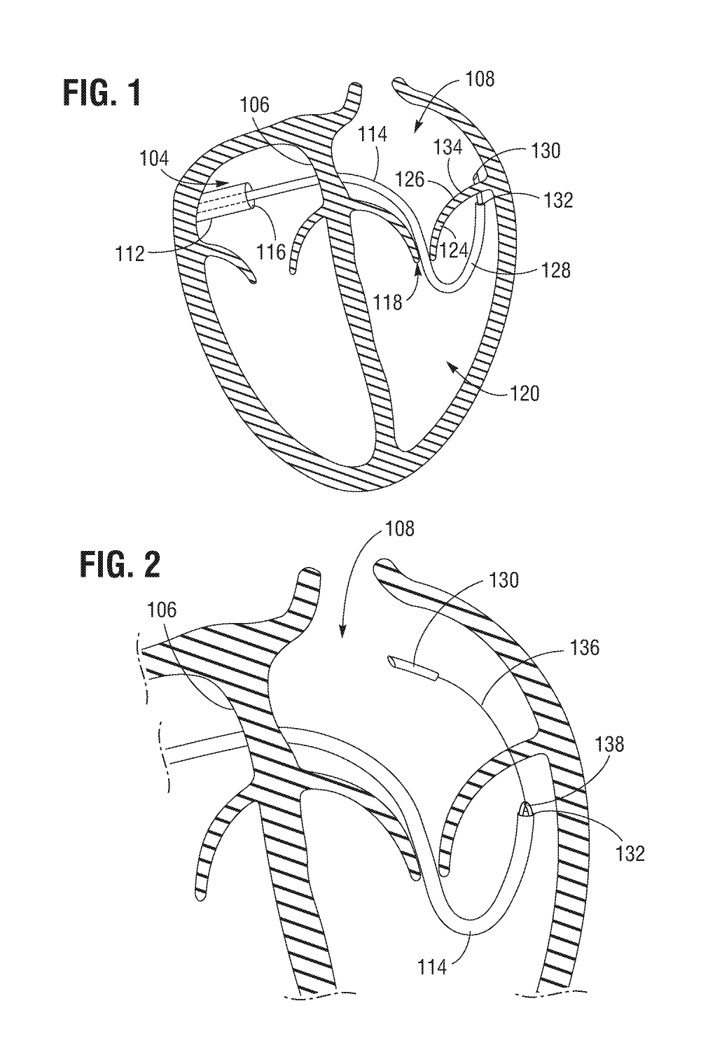

FIGS. 1-7 show an exemplary process for delivering an anchor device to the native posterior mitral valve leaflet transseptally, for example, from the right atrium 104, through the interatrial septum 106, and into the left atrium 108. As shown in FIG. 1, an outer, delivery catheter 112 can be inserted into the right atrium 104. The delivery catheter 112 can have a steering mechanism, such as one or more pull wires extending the length of the catheter, configured to adjust the curvature of the distal end portion of delivery catheter 112 to assist in steering the catheter through a patient's vasculature.

A deployment catheter 114 can be advanced through the delivery catheter 112 and extended from a distal opening 116 of the delivery catheter 112 into the right atrium 104. The deployment catheter 114 can then be inserted transseptally through the interatrial septum 106, anterogradley through the mitral valve 118 into the left ventricle 120, and then superiorly towards the inferior surface 124 of the posterior mitral valve leaflet 126. In particular examples, the deployment catheter 114 can be inserted through the interatrial septum 106 on, or in an area in proximity to, the fossa ovalis.

A distal end portion 128 of the deployment catheter 114 desirably is configured to form a 180-degree curve or bend so that it can be placed to extend through the mitral valve 118 and towards the inferior surface 124 of the posterior mitral valve leaflet 126, as shown in FIG. 1. The distal end portion 128 can be pre-formed (such as by heat shaping) to have a 180-degree curve in a non-deflected state. The pre-formed distal end portion 128 can be deflected to a non-curved, substantially straight configuration for advancement through the delivery catheter 112. When the distal end portion 128 is advanced from the distal opening 116 of the delivery catheter 112, the distal end portion 128 can revert back to the non-deflected, curved configuration. Alternatively, the deployment catheter 114 can be provided with a steering mechanism, such as a pull wire, that is configured to bend the distal end portion 128 from a straight configuration to the curved configuration shown in FIG. 1.

A needle 130 can be advanced from a distal opening 132 of the deployment catheter 114, into the inferior surface 124 of the posterior mitral valve leaflet 126, and out through the superior surface 134 of the posterior mitral valve leaflet 126. In some embodiments, the deployment catheter 114 and/or the delivery catheter 112 are sufficiently stiff to promote piercing of the posterior mitral valve leaflet 126. In particular aspects, the needle 130 can be omitted, and the deployment catheter 114 can penetrate the posterior mitral valve leaflet 126.

With reference to FIG. 2, the needle 130 can be further advanced from the distal end 132 of the deployment catheter 114 into the left atrium 108. The needle 130 can be coupled to the distal end of a tension member 136. The tension member 136 can be a length of material, for example, an elongate length of flexible material such as suture, cable, string, coil, cord, or wire. The proximal end of the tension member 136 can be further coupled to an anchor device 138. When the needle 130 and the tension member 136 are sufficiently withdrawn from the distal end 132 of the deployment catheter 114, the anchor device 138 can be withdrawn from the distal end 132 of the deployment catheter 114, proximate the inferior surface 124 of the posterior mitral valve leaflet 126. In a specific example, the anchor device 138 can be secured to the posterior mitral valve leaflet 126. In other embodiments, the tension member 136 can be passed through the tissue of the annulus immediately adjacent the posterior leaflet 126.

In at least some implementations, the anchor device 138 can be manipulable between a first configuration adapted to facilitate delivery and deployment of the anchor device to a second configuration configured to secure the anchor device relative to the posterior mitral valve leaflet 126. For example, the anchor device 138 can assume a folded configuration within the deployment catheter 114 and unfold once the anchor device has been withdrawn from the distal end 132 of the deployment catheter 114. In other implementations, the anchor device 138 can have the same configuration during both delivery and deployment. In some cases, the anchor device 138 may rotate after being deployed.

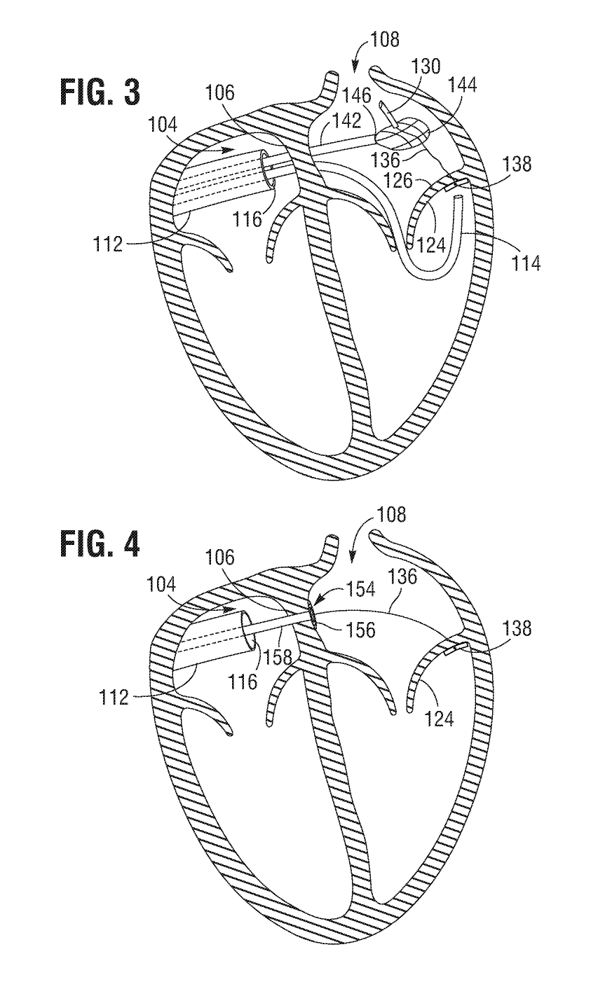

FIG. 3 illustrates a snare catheter 142 extending distally from the distal opening 116 of the delivery catheter 112. The snare catheter 142 can extend from the right atrium 104, through the interatrial septum 106, and into the left atrium 108. A snare member 144 can extend distally from the distal end 146 of the shaft of the snare catheter 142 and into the interior of the left atrium 108. The needle 130 can be captured using the snare member 144, and the needle 130, snare member 144, and a portion of the tension member 136 can be withdrawn into the delivery catheter 112 by retracting the snare catheter 142 back into the delivery catheter. In some implementations, the deployment catheter 114 can be withdrawn from the body, leaving the anchor device 138 in place against the inferior surface 124 of the posterior mitral valve leaflet 126.

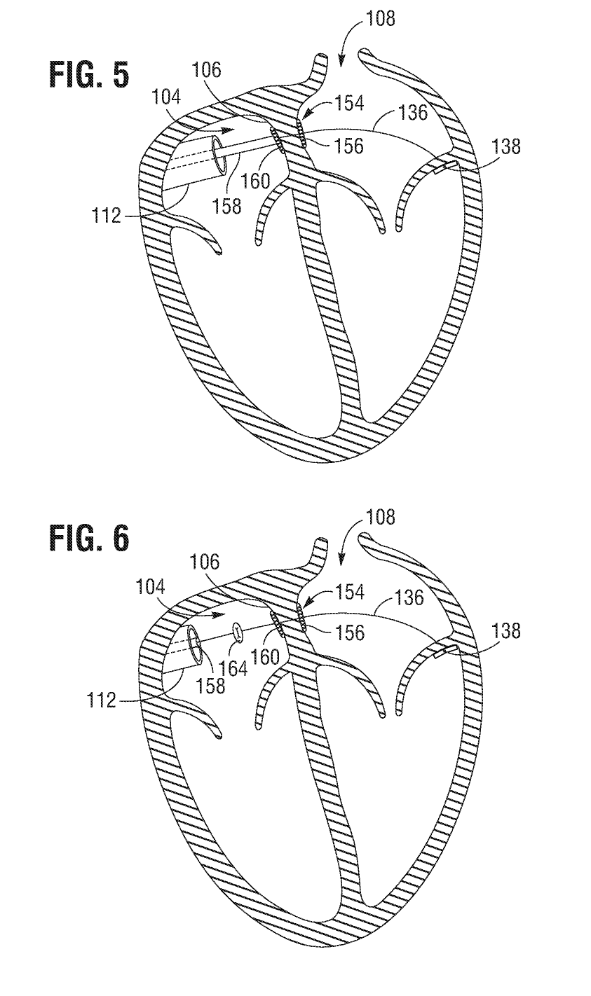

Referring now to FIG. 4, a left atrial portion or anchor 156 of a closure member 154 can be placed on the left atrial side of the interatrial septum 106. For example, the closure member 154 can be advanced over the tension member 136 within a closure device catheter 158 advanced through the delivery catheter 112. As shown in FIG. 5, the closure device catheter 158 can be withdrawn into the right atrium 104, and a right atrial portion or anchor 160 of the closure member 154 can be placed on the right atrial side of the interatrial septum 106. A central portion of the closure member 154 extends through the opening in the septum 106 and connects the left and right anchors 156, 160 of the closure member. In particular implementations, the closure member 154 can be a closure device suitable for closing a patent foramen ovale or an atrial septal defect, such as the HELEX® Septal Occluder (W. L. Gore & Associates, Flagstaff, Ariz.), the Amplatzer® Septal Occluder (St. Jude Medical, Inc., St. Paul, Minn.), the CardioSEAL® and STARFlex® Septal Occluders (NMT Medical, Inc., Boston, BA), the Sideris button and ButtonSeal COD devices, Das-Angel Wing™ Occlusion Device (Microvena, Vadnais, Minn.), and the ASD Occluder System (Osypka Corp, Germany).

FIG. 6 illustrates a fastener 164 (such as a suture clip or similar locking or retaining mechanism) that can be advanced over the tension member 136 and placed against the right atrial portion 160 of the closure member 154. Various suture clips and deployment techniques for suture clips that can be used in the methods disclosed in the present application are disclosed in U.S. Publication Nos. 2014/0031864 and 2008/0281356, and U.S. Pat. No. 7,628,797, which are incorporated herein by reference. In the case of a slidable fastener, the fastener 164 can be movable along the tension member 136 in a direction toward the interatrial septum 106, and configured to resist movement along the tension member 136 in the opposite direction. Additional fasteners suitable for use as a fastener 164, including lockable fasteners/fasteners that can allow the tension of the tension member 136 to be adjusted during deployment of the fastener 164, include fasteners described in U.S. Patent Publication No. 2012/0022633, incorporated by reference herein, for example, FIGS. 29-39 and paragraphs 227-237. The proximal end (the end opposite the end attached to the anchor device 138) of the tension member 136 can be looped, if appropriate, to facilitate deployment of the fastener 164.

In some implementations, the tension in the tension member 136 can be adjusted or maintained while the fastener 164 is being deployed, such as by pulling proximally on one or more ends of the tension member 136 located outside of the patient's body. In other implementations, the fastener 164 can allow the tension applied to the anchor device 138 to be adjusted after the fastener 164 is deployed. For example, the fastener 164 can employ a ratcheting type mechanism or other mechanism that allows the fastener 164 to be advanced over the tension member 136 in a first direction (e.g., distal), but not in the opposite direction. In further examples, the fastener 164 can be selectively lockable, such that fastener 164 can be unlocked in order to adjust its position/the tension in the tension member 136 and locked when a desired tension has been achieved. The tension member 136 can include surface features, such as ridges, grooves, or barbs, to facilitate tensioning and securing the tension member 136 to the fastener 164 in order to apply a desired tension to the anchor device 138.

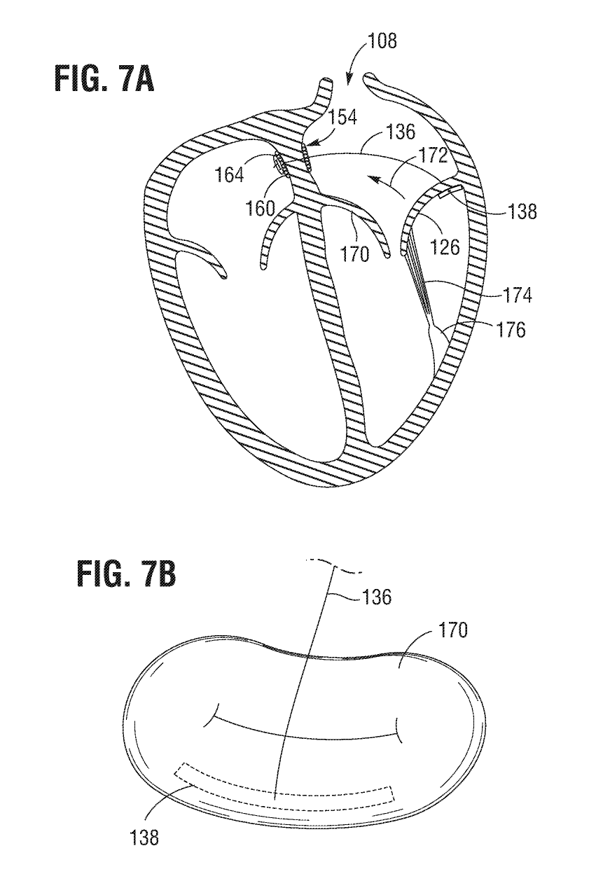

A remodeling force can be applied to the heart tissue by pulling the needle 130, or a portion of the tension member 136, proximally to remodel the heart tissue, such as reducing the distance between the interatrial septum 106 and the posterior mitral valve leaflet 126. With reference to FIG. 7A, a proximal force applied to the tension member 138 through the tension member 136 can cause the posterior mitral valve leaflet 126 to be pulled anteriorly and superiorly towards the interatrial septum 106, and improving coaptation between the posterior 126 and anterior 170 leaflets of the mitral valve 118.

As shown in FIG. 7B, the anchor device 138 desirably is positioned so as to extend along the length of the posterior leaflet 126 generally extending in a direction from one commissure to the other commis sure. In this manner, the remodeling force of the tension member 136 can be applied to the leaflet 126 substantially along its entire length. The remodeling force can therefore pull substantially the entire length of the leaflet 126 anteriorly and superiorly toward the septum 106 to improve coaptation with the anterior leaflet 170 along substantially the entire lengths of the coaptation edges of the native leaflets. As further shown in FIG. 7B, the anchor device 138 can be curved along its length so as to generally correspond to the shape of the annulus adjacent the posterior leaflet 126.

An excess portion of the tension member 136 extending from the fastener 164 into the right atrium 104 can be cut or severed. For example, a fastener deployment device associated with the fastener 164 can include a cutting element at its distal end that can be engaged by a clinician. Alternatively, a separate cutting device (e.g., a cutting catheter or a catheter having a controllable cutting element) can be inserted through the delivery catheter 112 (or otherwise inserted into the patient, and proximate the right atrial portion 160 of the closure member 154). With the anchor member 138 secured in place by the fastener 164 and the closure member 154, the delivery catheter 112 and its associated components can be withdrawn from the patient's body.

The tension member 136, secured by the fastener 164 bearing against the right atrial portion 160 of the closure member 154, places the anchor member 138 under tension. In particular, the tension member 136 can apply an anteriorly and superiorly directed force 172 that can pull the posterior mitral valve leaflet 126 toward the interatrial septum 106. This force 172 can pull the posterior mitral valve leaflet 126 towards, and promote coaptation with, the anterior mitral valve leaflet 170. In addition, the force 172 applied to the posterior mitral valve leaflet 126 can pull the chordae tendineae 174 and the papillary muscles 176 inwardly and upwardly toward the left atrium 108 toward their natural position beneath the commissures of the mitral valve leaflets 126, 170, thereby improving coaptation of the leaflets 126, 170, and reducing or preventing mitral regurgitation.

In particular embodiments, one or more of the deployment catheter 114, the snare catheter 142, the snare member 144, the tension member 136, the closure device catheter 158, the closure member 154, the fastener 164, and the anchor device 138 can be pre-loaded within the delivery catheter 112 and all components can be delivered into the heart together as a unit. Each component can then be advanced from the delivery catheter 112 in the sequence described above. Although shown as deployed with the delivery catheter 112 located in the right atrium 104, in some implementations, the delivery catheter 112 can be positioned within the left atrium 108 (such as through a transseptal puncture) for certain steps of the above-described method, such as the steps depicted in FIGS. 1-4. Similarly, although FIGS. 1-7 have been described as using a delivery catheter 112 that can include a deployment catheter 114, a snare catheter 142, and a closure device catheter 158, in some implementations, one or more of the deployment catheter 114, the snare catheter 142, and the closure device catheter 158 can be provided as a separate catheter (i.e., not located within, or advanced through, the delivery catheter 112).

Although the method illustrated in FIGS. 1-7 was described as including deploying an anchor device 138 below the posterior mitral valve leaflet 126, and inserting the tension member 136 through the leaflet, in other embodiments, the anchor device 138 can be deployed in a different manner. For example, rather than inserting the needle 130 through the inferior surface 124 of the posterior mitral valve leaflet 126, the deployment catheter 114 or the needle can be used to puncture the leaflet from the superior surface 134 to the inferior surface. The anchor device 138 can then be deployed through the deployment catheter 114, such as through the puncture in the leaflet 126, to a position beneath the leaflet. The anchor device 138 can then be secured in a manner analogous to the steps shown FIGS. 4-7.

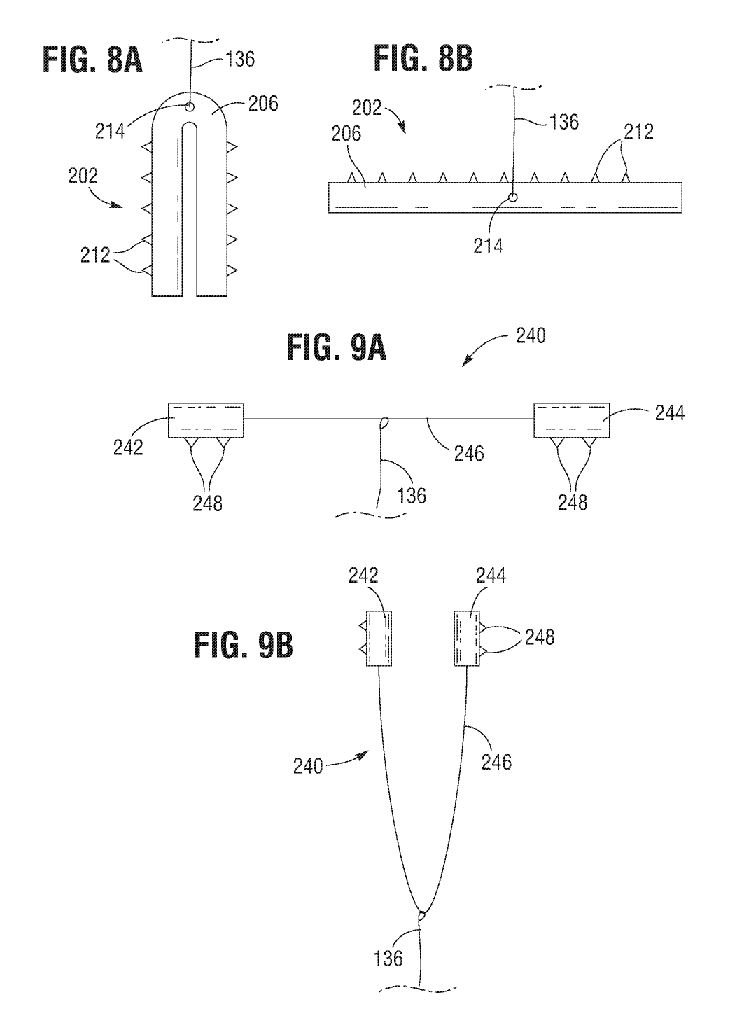

FIGS. 8A and 8B, and 9A and 9B, illustrate various anchor devices that may be used as the anchor device 138 in the method depicted in FIGS. 1-7, and can be attached to the tension member 136. Although the anchor devices are generally shown as having straight sides, it should be appreciated that other shapes can be used, including arcuate shapes. In particular, the anchor member 138 can be shaped to cooperate with native leaflet tissue.

The anchor device 202 of FIGS. 8A and 8B includes an elongate body 206. The body 206 can include one or more gripping elements 212, such as spikes or barbs, that can provide enhanced frictional engagement with a heart valve leaflet, such as the posterior mitral valve leaflet. The gripping elements 212 desirably have pointed ends that can penetrate the surface of the native leaflet to promote engagement of the body 206 with a native leaflet. In the illustrated embodiment, the body 206 has a plurality of gripping elements 212. In further embodiments, the body 206 can lack the gripping elements 212, can have a different number of gripping elements, or the gripping elements can be arranged in a different manner.

The body 206 can define an aperture 214, such as in the middle, longitudinally, of the body 206. A length of the tension member 136 can be inserted through the aperture 214 and an end secured about the aperture, such as by affixing it to the body 206 or by tying the end of the tension member 136 about the aperture 214. In other cases, the aperture 214 can be omitted and/or the tension member 136 affixed to the body 206 in another manner. The number and location of apertures or other attachment points for the tension member 136 can be varied, such as to more evenly distribute the tensioning force along the body 206 of the anchor device 202.

In use, the anchor device 202 can have a first configuration, shown in FIG. 8A, where the anchor device can be bent, such to more easily fit within the lumen of a delivery device, such as the deployment catheter 114. Once the anchor device 202 has been deployed, such as being advanced from the end of the deployment catheter 114, the anchor device can assume the configuration shown in FIG. 8B, where the anchor device has unfolded in order for a larger portion of the surface of the anchor device to bear against a native heart leaflet.

The anchor device 202 can be formed from a shape memory alloy (such as Nitinol or another nickel-titanium alloy). The anchor device 202 can be heat set such that the anchor device can be in the bent configuration shown in FIG. 8A during deployment. When the anchor device 202 is removed from a catheter inside the heart, the anchor device can assume its heat set, unbent position (FIG. 8B). In further examples, the anchor device 202 can be formed from a resilient material, such as a biocompatible polymer, that is capable of being deformed from the deployed configuration of FIG. 8B to the delivery configuration of FIG. 8A, and back.

In a further implementation, rather than being bendable, the tension member 136 can be at least substantially parallel to the longitudinal axis of the anchor device 202 while the anchor device 202 is being advanced through a catheter. When the anchor device 202 is withdrawn from the catheter proximate the posterior mitral valve leaflet, the anchor device 202 and tension member 136 can rotate relative to one another such that the tension member 136 is at least substantially perpendicular to the longitudinal axis of the anchor device 202.

FIGS. 9A and 9B illustrate an anchor device 240 having two anchor portions 242, 244 coupled by an elongate connecting member 246. The connecting member 246 can be, for example, a length of wire or cable. Each of the anchor portions 242, 244 can include one or more gripping elements 248. In some cases, one or both of the anchor portions 242, 244 can lack gripping elements 248, or the gripping elements can be distributed in a manner other than that shown.

The tension member 136 can be coupled to the connecting member 246, such as at least about approximately the midpoint between the anchor portions 242, 244. During delivery, the tension member 136 can be at least substantially parallel to the longitudinal axis of the connecting member 246. After the anchor member 240 is removed from a catheter, the tension member 136 can rotate relative to the anchor member 240 such that the tension member 136 is at least substantially perpendicular to the connecting member 246.

In another aspect, the anchor device 240 can be inserted into a catheter in a folded state, as shown in FIG. 9B, such that the anchor portions 242, 244 are adjacent each other within the catheter. In particular examples, the connecting member 246 of the anchor device 240 can be formed from a shape memory alloy (such as Nitinol or another nickel-titanium alloy). The connecting member 246 can be heat set such that when the anchor device 240 is deployed from a catheter, the connecting member 246 can assume a linear configuration (FIG. 9A) and the anchor portions 242, 244 can be pulled against the inferior surface of the posterior mitral valve leaflet. In another implementation, the anchor device 240 can be formed from a resilient material, such as a biocompatible polymer, that can be compressed from the configuration of FIG. 9A to the configuration of FIG. 9B for delivery/deployment, and then reassume the configuration of FIG. 9A after deployment in the heart.

When the tension member 136 is inserted through the leaflet, and placed under tension, the anchor portions 242, 244 can abut the inferior surface of the posterior mitral valve leaflet. The use of spaced-part anchor members 242, 244 can help distribute a remodeling force along the inferior surface of the posterior mitral valve leaflet.

Other devices may be used as the anchor device 138. In some cases, the anchor device 138 can be formed from a disc of material, such as a disc of braided wire or a polymer disc. In other embodiments, the anchor device 138 can be an inflatable balloon. In some cases, the balloon can be relatively non-elastic, such that it can be inflated to a fixed degree, and thus have a fixed shape and size. In other cases, the balloon can be made from a relatively compliant and/or elastic material, allowing it to be inflated to different levels, and thus different sizes and/or shapes.

In a particular example, all or a portion of a septal closure device (such as closure device used for closing a patent foramen ovale or an atrial septal defect) can be used as the anchor device 138. Suitable septal closure devices can include those described for use as the closure member 154 of FIG. 4. The septal closure device, when used to close a septal defect, can include a portion to be deployed in the right atrium and a portion to be deployed in the left atrium. When used as an anchor device 138, in one implementation, the left or right atrial portion can be deployed beneath the posterior mitral valve leaflet 126. In another implementation, one of the atrial portions can be disposed beneath the posterior mitral valve leaflet and the other atrial portion can be disposed above the superior surface of the leaflet (e.g., an anchor device 138 can comprise a first portion 156 mounted on the superior surface of the leaflet and a second portion 160 mounted on the inferior surface of the leaflet).

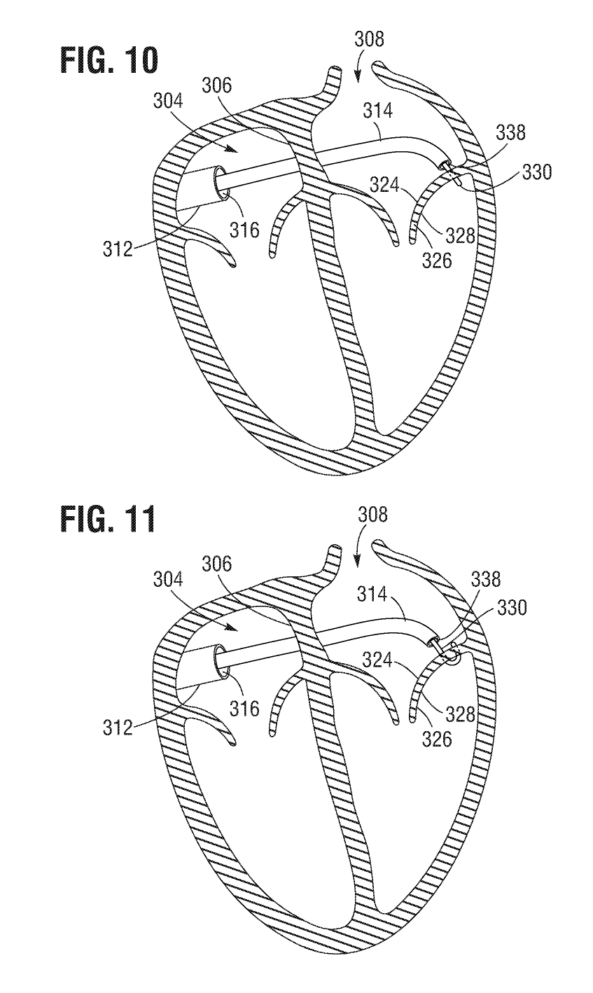

FIGS. 10-16 show an exemplary process for applying a remodeling force to the heart to improve coaptation of the mitral valve leaflets using a tension member. The process can be generally similar to the process described with respect to FIGS. 1-7, but a separate anchor device need not be used to secure the tension member to a native leaflet. The tension member can be delivered transseptally, for example, from the right atrium 304, through the interatrial septum 306, and into the left atrium 308. As shown in FIG. 10, an outer, delivery catheter 312 can be inserted into the right atrium 304. The delivery catheter 312 can have a steering mechanism, such as one or more pull wires extending the length of the catheter, configured to adjust the curvature of the distal end portion of delivery catheter 312 to assist in steering the catheter through a patient's vasculature.

A deployment catheter 314 can be advanced through the delivery catheter 312 and extended from a distal opening 316 of the delivery catheter 312 into the right atrium 304. The deployment catheter 314 can then be inserted transseptally through the interatrial septum 306 into the left atrium 308, and then inferiorly towards the superior surface 324 of the posterior mitral valve leaflet 326. In particular examples, the deployment catheter 314 can be inserted through the interatrial septum 306 on, or in an area in proximity to, the fossa ovalis. A needle 330 can be advanced from a distal opening 338 of the deployment catheter 314. The needle 330 can penetrate the posterior mitral valve leaflet 326, extending through the inferior surface 328 of the posterior mitral valve leaflet 326.

With reference to FIG. 11, the needle 330 can be directed superiorly to again penetrate through the posterior mitral valve leaflet 326, out through the superior surface 324 of the posterior mitral valve leaflet, and into the left atrium 308. The needle 330 can include a preset bend, such that the needle 330 curves as it is extended out of the distal opening 338 of the deployment catheter 314. As the needle 330 curves, the tip of the needle can be oriented towards the inferior surface 328 of the posterior mitral valve leaflet 326. In particular implementations, the needle 330 can be made from a shape memory alloy (such as Nitinol or another nickel-titanium alloy) and heat set to produce the desired degree of bending when the needle 330 is extended from the distal opening 338 of the deployment catheter 314.

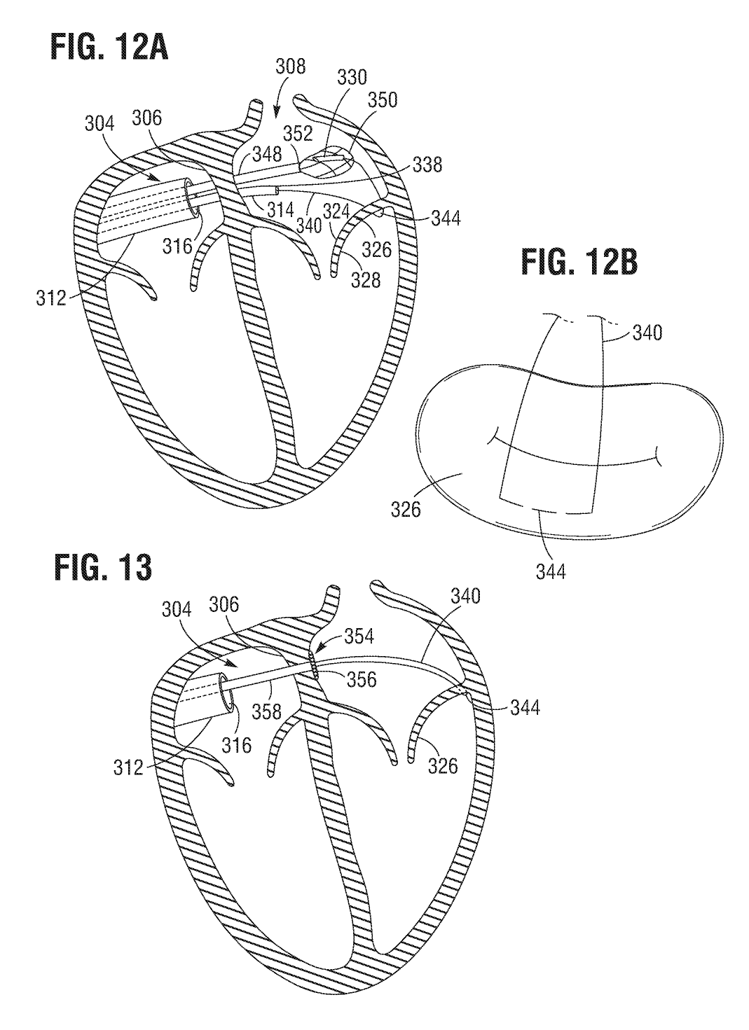

With reference to FIG. 12A, the needle 330 can be coupled to a distal end of a tension member 340 that can extend proximally through the distal opening 338 of the deployment catheter 314. The tension member 340 can be a length of material, for example, an elongate length of flexible material such as suture, cable, string, coil, cord, or wire. In particular examples, the tension member 340 can be advanced through the needle 330, or the distal end of the tension member 340 can be coupled to the needle 330. In other cases, the needle 330 can be omitted, and the tension member 340 can be advanced directly from the deployment catheter 314.

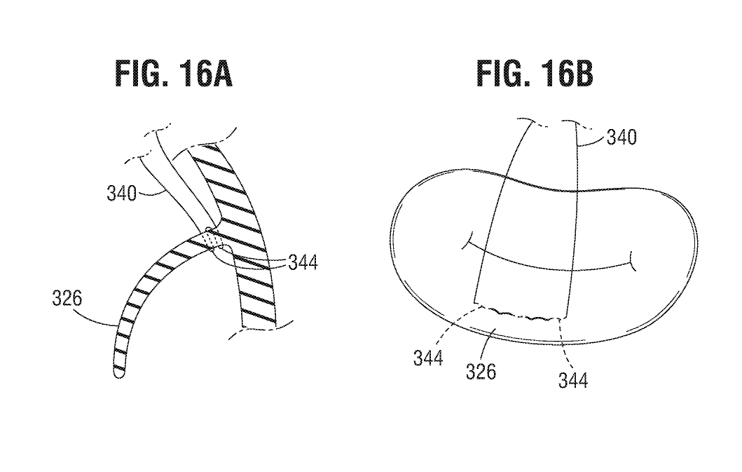

The deployment catheter 314 can be withdrawn, passing back through the inferior surface 328 and out the superior surface 324 of the posterior mitral valve leaflet 326. The deployment catheter 314 can be further withdrawn into the left atrium 308, as shown in FIG. 12A. After removal of the delivery catheter 314, a loop or stich 344 of the tension member 340 is left underneath the posterior mitral valve leaflet 326 (as shown in FIGS. 12A and 12B). As shown in FIG. 12B, the loop or stitch 344 can be formed along the length, or perimeter, of the leaflet 326 in a direction extending generally from one commissure to the other commissure. In other cases, the loop or stich 344 can be disposed in another direction, such as along the width of the leaflet 326 in a direction extending generally from the free edge of the leaflet to the annulus. Although a single loop of the tension member 344 is shown, in at least some implementations, the deployment catheter 314 can pass multiple times through the posterior mitral valve leaflet 326, forming a plurality of loops 344 of the tension member 340 (e.g., FIGS. 16A and 16B).

With continued reference to FIG. 12A, a snare catheter 348 can extend distally from the distal opening 316 of the delivery catheter 312. The snare catheter 348 can extend from the right atrium 304, through the interatrial septum 306, and into the left atrium 308. A snare member 350 can extend distally from the distal end 352 of the shaft of the snare catheter 348 and into the interior of the left atrium 308. The free end of the tension member 340 (including the needle 330, when used with the tension member 340) can be captured using the snare member 350, and the snare member 350 and the free end of the tension member 340 can be withdrawn into the delivery catheter 312 by retracting the snare catheter 348. In some implementations, the deployment catheter 314 can be withdrawn from the body, leaving the loop 344 in place against the inferior surface 328 of the posterior mitral valve leaflet 326.

With reference to FIG. 13, a left atrial portion or anchor 356 of a closure member 354 can be placed on the left atrial side of the interatrial septum 306. For example, the closure member 354 can be advanced over the tension member 340 inside of a closure device deployment catheter 358 that is advanced through the delivery catheter 312.

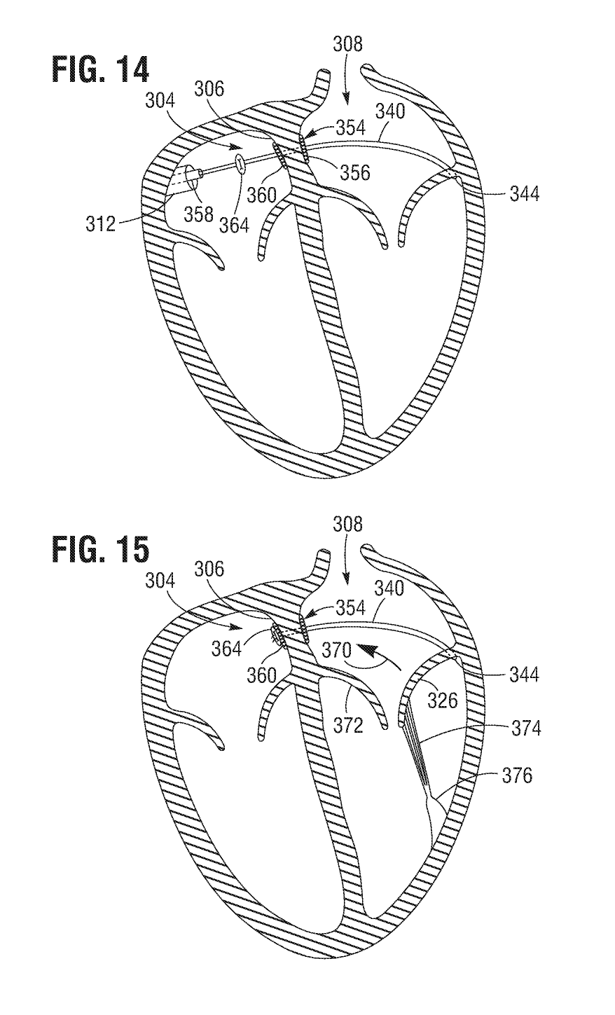

As shown in FIG. 14, the closure device deployment catheter 358 can be withdrawn into the right atrium 304, and a right atrial portion or anchor 360 of the closure member 354 can be placed on the right atrial side of the interatrial septum 306 with a central portion of the closure member extending through the septum. In particular implementations, the closure member 354 can be any of various suitable closure devices described above in connection with the closure member 154.

FIG. 15 illustrates a fastener 364 (such as a suture clip or similar locking or retaining mechanism) that can be advanced over the tension member 340 and placed against the right atrial portion 360 of the closure member 354. The fastener 364 can be any of various suitable fasteners described above in connection with the fastener 164. In some implementations, the tension in the tension member 340 can be adjusted or maintained while the fastener 364 is being deployed, such as by pulling proximally on one or more ends of the tension member 340 located outside of the patient's body. In other implementations, the fastener 364 can allow the tension applied to the loop 344 to be adjusted after the fastener 364 is deployed. For example, the fastener 364 can employ a ratcheting type mechanism or other mechanism that allows the fastener 364 to be advanced over the tension member 340 in a first direction (e.g., distal), but not in the opposite direction. In further examples, the fastener 364 can be selectively lockable, such that the fastener 364 can be unlocked in order to adjust its position/the tension in the tension member 340 and locked when a desired tension has been achieved. The tension member 340 can include surface features, such as ridges, grooves, or barbs, to facilitate tensioning and securing the tension member 340 to the fastener 364 in order to apply a desired tension to the loop 344.

A remodeling force 370 can be applied to the heart tissue by pulling the ends of the tension member 340 proximally to remodel the heart tissue, such as reducing the distance between the interatrial septum 306 and the posterior mitral valve leaflet 326. In particular, a proximal force applied to the loop 344 through the ends of the tension member 340 can cause the posterior mitral valve leaflet 326 to be pulled anteriorly and superiorly towards the interatrial septum 306, improving coaptation between the posterior 326 and anterior 372 leaflets. In addition, the force applied to the posterior mitral valve leaflet 326 can pull the chordae tendineae 374 and the papillary muscles 376 inwardly and upwardly toward the left atrium 308 toward their natural position beneath the commissures of the mitral valve leaflets 326, 372, thereby improving coaptation of the leaflets 326, 372, and reducing or preventing mitral regurgitation.

With reference to FIG. 15, an excess portion of the tension member 340 extending from the fastener 364 into the right atrium 304 can be cut or severed. For example, a fastener deployment device associated with the fastener 364 can include a cutting element at its distal end that can be engaged by a clinician. Alternative, a separate cutting device (e.g., a cutting catheter or a catheter having a controllable cutting element) can be inserted through the delivery catheter (or otherwise inserted into the patient, and proximate the right atrial portion 360 of the closure member 354). With the loop 344 secured in place by the fastener 364 and the closure member 354, the delivery catheter 312 and its associated components can be withdrawn from the patient's body.

In particular embodiments, one or more of the deployment catheter 314, the snare catheter 348, the snare 350, the closure device deployment catheter 358, the closure device 354, the fastener 364, and the tension member 340 can be pre-loaded within the delivery catheter 312 and all components can be delivered into the heart together as a unit. Each component can then be advanced from the delivery catheter 312 in the sequence described above. Although shown as deployed with the delivery catheter 312 located in the right atrium 304, in some implementations, the delivery catheter 312 can be positioned within left atrium 308 (such as through a transseptal puncture) for certain steps of the above-described method, such as the steps depicted in FIGS. 10-15. Similarly, although FIGS. 10-15 have been described as using a delivery catheter 312 that can include a deployment catheter 314, a snare catheter 348, and a closure device deployment catheter 358, in some implementations, one or more of the deployment catheter 314, the snare catheter 348, and the closure device deployment catheter 358 can be provided as a separate catheter (i.e., not located within, or advanced through, the delivery catheter 312).

FIGS. 16A and 16B illustrate the result of forming multiple loops or stitches 344 of the tension member 340 through the leaflet 326 during the method depicted in FIGS. 10-15. As shown in FIG. 16A, the loops or stitches 344 can be formed along the width of the leaflet in a direction extending generally from the free edge of the leaflet to the annulus. As shown in FIG. 16B, the loops or stitches 344 can be formed along the length, or perimeter, of the leaflet in a direction extending generally from one commissure to the other commissure.

In alternative embodiments, one or both ends of a tension member 136, 340 can be secured to the septum without a closure member 154, 354 implanted in the septum 106, 306. For example, one or both ends of the tension member 136, 340 can be retracted through the opening in the septum and secured with a fastener 164, 364 that can bear directly against the surface of the septum in the right atrium.

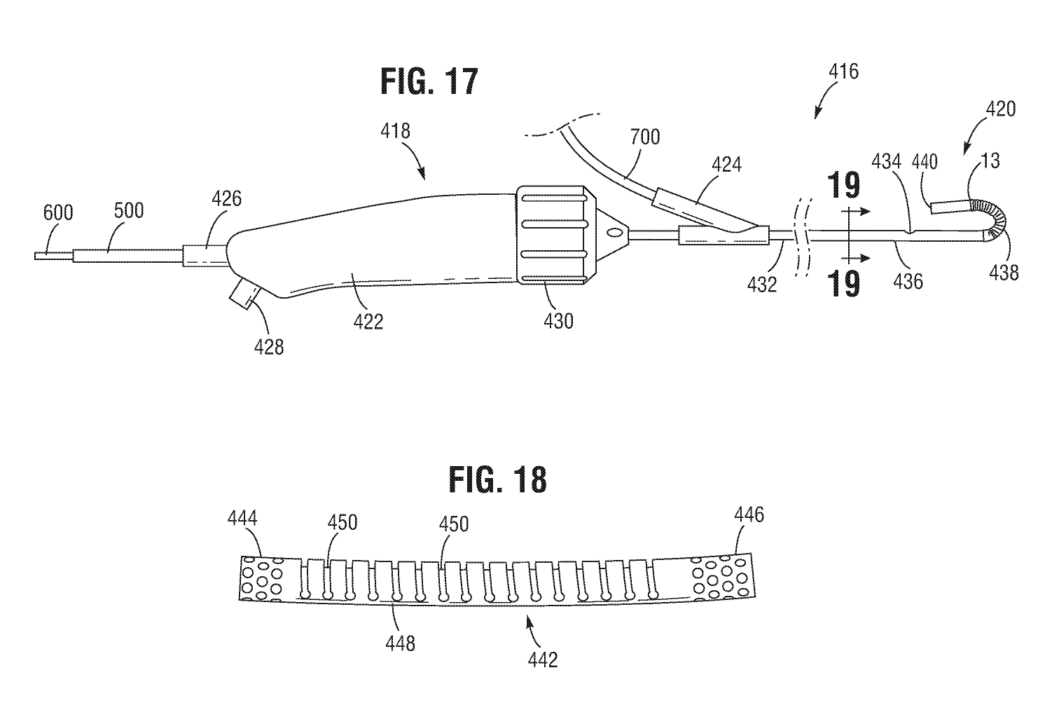

FIGS. 17-25 illustrate an embodiment of a tension member delivery assembly and methods to deploy a tension member, and optionally other components (e.g., an anchor device, closure member, or fastener) to the heart. The tension member delivery assembly in the illustrated embodiment generally comprises a steerable delivery catheter 416, a deployment catheter 500, a needle wire 600, and a snare catheter 700.

The delivery assembly of FIGS. 17-25 can be used to implant a tension member through a native leaflet as shown in FIGS. 1-6 and 10-15. For example, the steerable delivery catheter 416, the deployment catheter 500, and the needle wire 600 can be used to perform the functions of the deployment catheter 114 described above to penetrate and place a tension member through the native posterior leaflet, and the snare catheter 700 can be used to perform the functions of the snare catheter 148, as shown in FIGS. 1-6. Similarly, the steerable delivery catheter 416, the deployment catheter 500, and the needle wire 600 can be used to perform the functions of the deployment catheter 314 described above to penetrate and place a tension member through the native posterior leaflet, and the snare catheter 700 can be used to perform the functions of the snare catheter 348, as shown in FIG. 10-15.

FIG. 17 shows an embodiment of the steerable catheter 416. The steerable catheter 416, or components thereof, is configured to extend into the left ventricle and deliver a tension member to an area below the posterior mitral valve leaflet, such as for use in an above-described method. The steerable catheter 416 comprises a proximal end portion 418 and a distal end portion 420. The proximal end portion 418 of the steerable catheter 416 can comprise a handle 422, from which a shaft 432 extends. Mounted on the shaft 432 adjacent the handle 422 is an entry port, such as in the form of a y-connector 424 that is in communication with a side opening in the shaft and a respective lumen in the shaft. The y-connector 424 can be used to allow insertion of other tools, for example a snare catheter 700 or guide wire, into the steerable catheter, as further described below.

The handle 422 can also include a plurality of other access ports, for example, ports 426 and 428 extending from the proximal end of handle 422. The access ports 426, 428 allow other tools or catheters to be inserted in lumens in the shaft 432. For example, as shown in FIG. 17, the deployment catheter 500 can be inserted into and through the steerable catheter 416 via the access port 426 and the needle wire 600 can be inserted into and through a respective lumen of the deployment catheter. The handle 422 of the steerable catheter 416 can further comprise an adjustment mechanism 430 configured to adjust the curvature of a steerable section 438 of the shaft 432, as further described below.

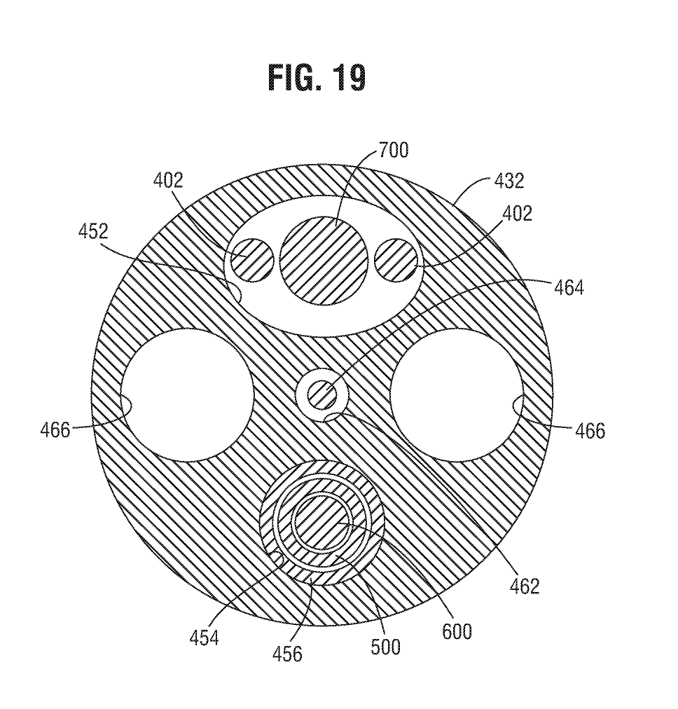

FIG. 19 shows a cross-sectional view of the shaft 432, according to one embodiment. In the illustrated embodiment, the shaft 432 has five lumens, including a first side lumen 452, a second side lumen 454, third and fourth side lumens 466, and a central lumen 462. The first side lumen 452 (also referred to as a “snare-catheter lumen”) is sized and shaped to receive the snare catheter 700 and one or more sections of a tension member 402 (such as the tension member 136 described in the method depicted in FIGS. 1-6 or the tension member 340 described in the method depicted in FIGS. 10-15). As shown, the snare-catheter lumen 452 can have an oval cross-sectional shape (in a plane perpendicular to the length of the shaft 432) to better accommodate the snare catheter 700 and the tension member 402. Although the snare-catheter lumen 452 is shown with two sections of a tension member 402, in other cases the snare-catheter lumen 452 can include a different number of sections of the tension member 402, including no sections, one section, or a plurality of sections. The snare-catheter lumen 452 has a proximal end in communication with the entry port 424 and a distal end in communication with a side opening 434 formed in the distal end portion of the shaft 432 (FIG. 17).

The second side lumen 454 desirably extends the entire length of the shaft 432 and has a proximal end in communication with the entry port 426 and a distal end forming a distal opening at the distal end of the shaft 432. Thus, as can be seen in FIGS. 17 and 19, the deployment catheter 500 can be inserted into the entry port 426 and advanced through the lumen 454, and the needle wire 600 can be inserted into and advanced through the lumen of the deployment catheter 500. The lumen 454 can have an inner liner 456 that desirably extends the entire length of the shaft 432. The inner liner 456 can comprise, for example, a braid reinforced polymer extrusion having one or more extruded layers. The reinforcing braid can be a braided sleeve (e.g., a braided metal sleeve) extending coaxially over the one or more extruded layers. In one specific implementation, the inner liner 456 comprises a nylon 12 outer extrusion, a Pebax® inner extrusion, and a braided stainless steel sleeve extending over the outer extrusion, although other suitable materials can be used. The outer surface of the inner liner 456 can be fixedly secured to the inner surface of the lumen 454, such as with a suitable adhesive.

The central lumen 462 serves as a pull wire lumen that allows passage of a pull wire 464. The third and fourth side lumens 466 can be open lumens or “dummy” lumens, which can extend along diametrically opposing sides of the central lumen 462. The lumens 466 can be potted, or otherwise sealed, to maintain hemostasis. Alternatively, one or both lumens may be used to pass a guide wire or other tool into the shaft 432. The lumens 466 can aid in providing uniform stiffness about the central axis of the shaft 432, which in turn provides for a smoother torque response of the shaft when it is torqued while in a deflected state.

The pull wire 464 has a proximal end operatively connected to the adjustment mechanism 430 and a distal end fixed within the shaft 432 at a distal end 468 of the steerable section 438. The adjustment mechanism 430 is configured to increase and decrease tension in the pull wire 464 to adjust the curvature of the steerable section 438 of the shaft 432. For example, rotating the adjustment mechanism 430 in a first direction (e.g., clockwise) increases the tension in the pull wire 464, which causes the steerable section 438 to bend or deflect into a curved configuration (as shown in FIG. 17). Rotating the adjustment mechanism in the opposite direction (e.g., counter-clockwise) reduces tension in the pull wire 464, which allows the steerable section 438 to return to its non-deflected configuration under its own resiliency. In the illustrated configuration, as shown in FIG. 17, the steerable section 438 can bend 180 degrees to permit navigation around the posterior leaflet and positioning of the distal end 440 of the shaft 432 at the superior or inferior surfaces of the posterior mitral valve leaflet, such as in carrying out an above-described method.

The steerable section 438 can be constructed from a relatively more flexible material than the portion of the shaft proximal of the steerable section or otherwise can be constructed to be relatively more flexible than the portion of the shaft proximal to the steerable section. In this manner, the curvature of the proximal portion can remain substantially unchanged when the curvature of the steerable section is adjusted by application of tension from the pull wire 464. Further details of the construction of the handle and the adjustment mechanism are described in U.S. Patent Application Publication Nos. 2013/0030519, 2009/0281619, 2008/0065011, and 2007/0005131, which are incorporated herein by reference.

The steerable section 438 can comprise a slotted metal tube 442 (FIG. 18) covered by a polymer sleeve or outer layer. As shown in FIG. 18, the slotted tube 442 in the illustrated configuration comprises a proximal end portion 444, a distal end portion 446, an intermediate portion 448 extending between the proximal and distal end portions, and a plurality of circumferentially extending, axially-spaced slots 450 formed in the intermediate portion 448, which impart flexibility to the steerable section 438. The tube 442 can be made of Nitinol or another suitable biocompatible metal with sufficient stiffness. The tube 442 can be formed, for example, by laser-cutting the slots 450 in a tubular piece of metal. The distal end of the pull wire 464 can be affixed to the distal end portion 446 of the tube, such as by welding. Except where the distal end of the pull wire 464 is affixed to the distal end portion 446, the pull wire can be “free-floating” within the much larger lumen of the tube 442, meaning that the pull wire can easily slide relative to the inner surface of the lumen with minimal friction, thereby preventing, or at least minimizing, kinking of the pull wire.

A conventional steerable catheter has a pull wire located within a pull wire lumen that is offset to one side of the central longitudinal axis of the catheter. A drawback of this design is that the catheter suffers from a phenomenon known as “whipping” when it is torqued or rotated relative to its central longitudinal axis to adjust the rotational position of the distal end portion of the catheter while it is in a contoured configuration following the contour of the anatomical pathway through which the catheter extends. When the catheter is rotated in this contoured configuration, the pull wire exerts uneven forces along the length of the delivery device, which causes the delivery device to become unstable and spring back to its non-torqued, low energy state.

As noted above, the pull wire 464 extends through a centrally located lumen 462 that extends along the central longitudinal axis of the shaft 432. Advantageously, placing the pull wire 464 in a centrally located lumen prevents the so-called “whipping” phenomenon of the shaft when a torqueing force is applied to shaft, allowing for controlled 360-degree torqueing of the shaft 432; that is, the distal end of the shaft can be rotated relative to the central longitudinal axis to any position through 360 degrees in three-dimensional space.

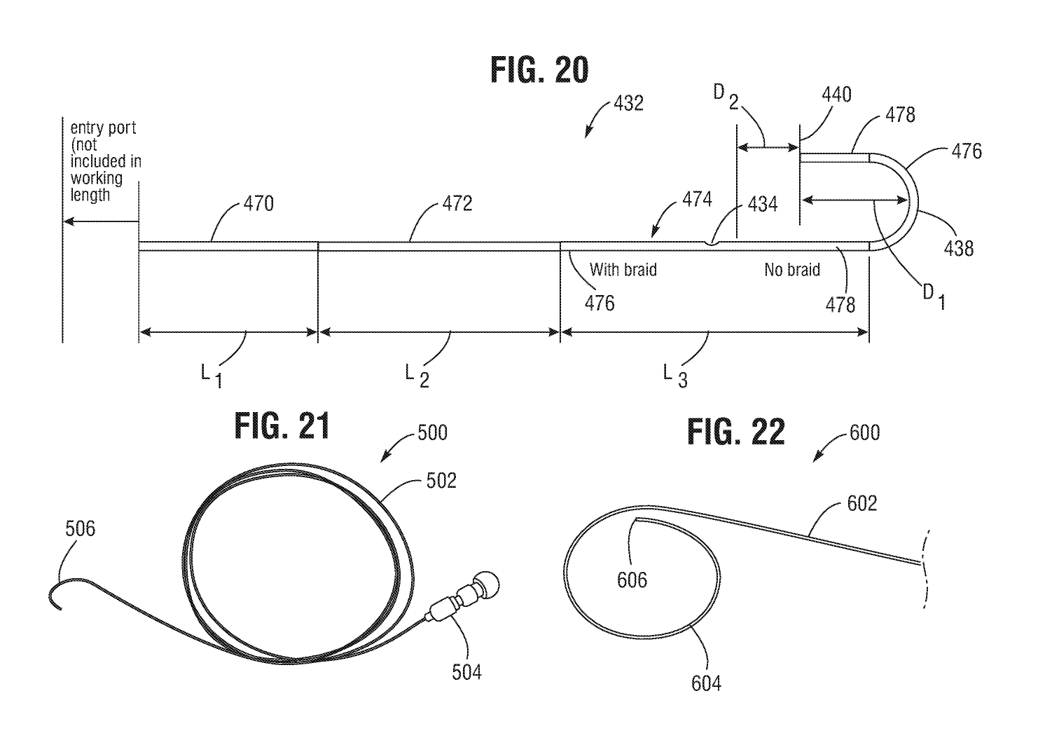

FIG. 20 shows details of the construction of a specific implementation of the shaft 432. In the illustrated configuration, the shaft 432 comprises a first section 470, a second section 472, a third section 474, and a fourth section 476. The fourth section 476 includes a steerable section 438 and a tip portion 478 distal to the steerable section. The first section 470 can be connected to the handle 422 (not shown in FIG. 20). The first section 470 has a length L₁, which can vary depending on a patient's height or point of vascular access. The first section 470 can comprise a polymer extrusion formed from one or more layers of different material. In a specific implementation, for example, the first section 470 comprises an inner layer made of nylon or ProPell and an outer layer made of 72D Pebax® or ProPell.

The second section 472 has a length L2, which in certain embodiments can be approximately 10-12 cm. The second section 472 can comprise a polymer extrusion formed from one or more layers of different material. In a specific implementation, for example, the second section 472 comprises an inner layer made of 72D Pebax® or ProPell and an outer layer made of 72D Pebax® or ProPell.

The third section 474 has a length L₃, which in certain embodiments can be approximately 8 cm. The third section 474 can comprise a polymer extrusion formed from one or more layers of different material. In a specific implementation, for example, the third section 474 comprises an inner layer made of 55D Pebax® or ProPell and an outer layer made of 55D Pebax® or ProPell.

The shaft 432 can further comprise a braided outer layer or sleeve extending over one or more of the first, second, and third sections 470, 472, 474, respectively. In particular embodiments, the braided layer extends over the entire length of the first and second sections 470, 472, and extends over the third section 474 from a first location where the third section is connected to the second section to a second location just proximal to the opening 434. Thus, the third section 474 can be subdivided into a braided section 476 and an unbraided section 478. The braiding can comprise, for example, 304V stainless steel wire, with dimensions of approximately 1 mil by 5 mil. The braid can have sixteen carriers, with fifty-five picks per inch (PPI), in a standard 1-over-2-under-2 pattern. In alternative embodiments, the braided layer can extend the entire length or substantially the entire length of the shaft 432.

The steerable section 438 can comprise a slotted metal tube 442 (FIG. 18) and an outer sleeve or jacket made of, for example, 32D Pebax® or ProPell. In particular embodiments, the steerable section 438 has a bend radius of approximately 10-14 mm, and can bend up to at least 180 degrees. The outer jacket of the steerable section 438 can be corrugated or ridged to facilitate bending. When the steerable section 438 is fully deflected such that the tip portion 478 extends substantially parallel to the third section 474, the distance D₁ from the distal most location of the steerable section 438 to the distal end 440 of the shaft can be approximately 2 cm. The longitudinal spacing between the distal end 440 of the shaft and the side opening 434 extends a distance D₂, which can approximately 1 cm.

FIG. 21 shows a deployment catheter 500, according to one embodiment, which is configured to cross or extend through a native leaflet or the annulus of the mitral valve for subsequent placement of the tension member 402. The deployment catheter 500 comprises an elongated shaft 502 that can have a lumen extending along its length for receiving the needle wire 600. The deployment catheter 500 can further include a leur fitting 504 connected to the proximal end of the shaft to facilitate insertion of the needle wire 600 into the lumen of the shaft. The fitting 504 can also be configured to lock or retain the needle wire 600 in place relative to the deployment catheter 500. The shaft 502 desirably has a pre-shaped or pre-curved distal end portion 506, which helps prevent or minimize kinking as it is advanced through the steerable section 438 of the steerable catheter when the steerable section is placed in the curved configuration.

In particular embodiments, the shaft 502 of the deployment catheter 500 has an outside diameter of about 0.27 inch, an inner diameter (the diameter of the lumen) of about 0.18 inch, and an overall length of about 69 inches or greater. The shaft 502 can comprise a polymer extrusion of one or more layers and can have a braided sleeve or outer layer extending over the extrusion. In one specific implementation, shaft 502 can comprise a multilayer extrusion comprising an inner layer made of ProPell, an intermediate layer made of nylon 12, and an outer layer made of ProPell. In an alternative implementation, the extrusion comprises a PTFE inner layer and the outer layer can contain barium sulfate. The barium sulfate can provide contrast during fluoroscopy. The braided outer sleeve can be similar to the braiding described above in connection with the shaft 432 of the steerable catheter, except that the deployment catheter shaft 502 desirably is stiffer. Thus, for example, a 5 mil by 25 mil 304V stainless steel wire can be used to form the braid. The braid PPI can be approximately 80-90. The distal end portion 506 can be pre-curved to a diameter of about 1 inch.

FIG. 22 shows an example embodiment of a needle wire 600 for puncturing a native leaflet or the annulus of the mitral valve. The needle wire 600 comprises a proximal portion 602, a distal portion 604, and a sharpened tip 606 configured to puncture native tissue, such as the annulus or a leaflet. The proximal portion 602 can be substantially straight in an un-deflected state and the distal portion 604 can be curved in an un-deflected state. The distal portion 604 can be, for example, shape-set or pre-curved to form a 360-degree curve having a diameter of, for example, about 19 mm. The overall length of the needle wire 600 is preferably longer than the deployment catheter 500 to allow for insertion and manipulation. In one specific implementation, the needle wire 600 has a length greater than 75 inches, is made of solid Nitinol, and has an outside diameter of approximately 0.16 inch to allow for insertion through the deployment catheter 500.

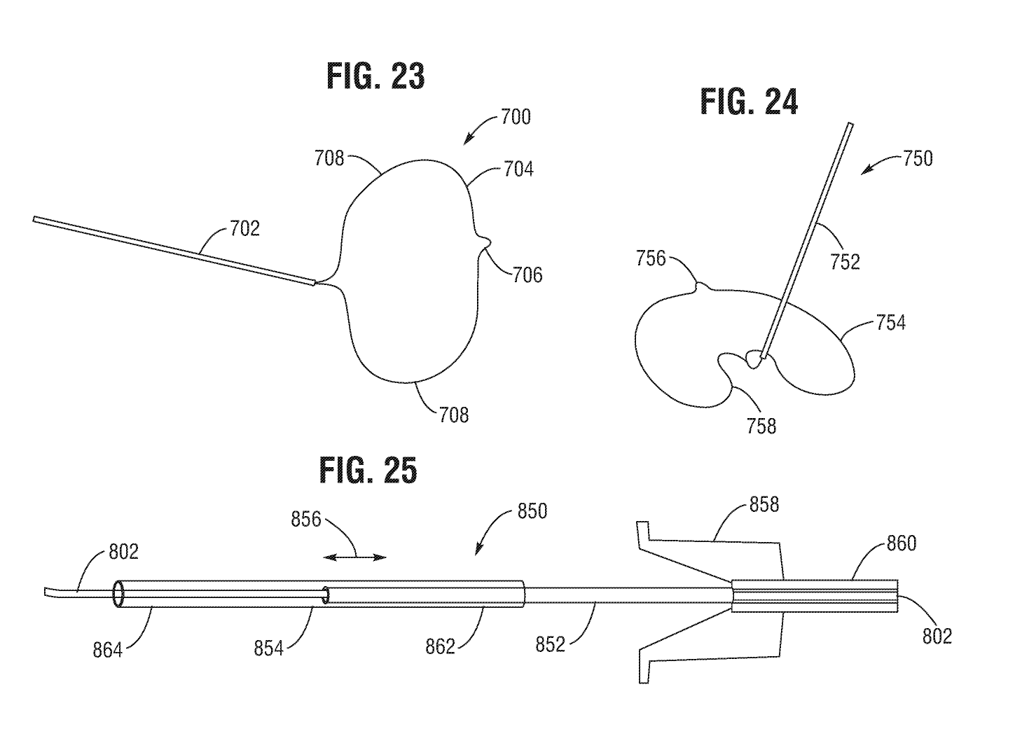

FIGS. 23 and 24 show different embodiments of a snare catheter that can be used for capturing an end of the tension member 402 once it is passed through a leaflet. FIG. 23 shows an embodiment of a snare catheter 700 comprising an elongated shaft 702 and a snare loop 704 extending from the distal end of the shaft 702. The snare loop 704 is radially expandable from a collapsed delivery state to an expanded, functional state (shown in FIG. 23) for capturing the end of the tension member 402 (or a needle coupled thereto). In the delivery state, opposite sides 708 of the loop 704 are compressed toward each other such that the sides 708 are generally straight and are in close proximity to each other such that the snare catheter 700 can be advanced through the lumen 452 of the steerable catheter 416. When the snare loop 704 is advanced from the distal opening 434 of the steerable catheter 416, the snare loop 704 can expand to its functional size for capturing the tension member 402, as further described below.

The snare loop 704 can extend from the shaft 702 at an angle less than 180 degrees, such as a 90-degree angle, to facilitate placement of the snare loop at a desired position inside the heart when capturing the tension member 402. The snare loop 704 can be generally oval in shape and can have a radially protruding section 706 diametrically opposed to the location where the loop is attached to the shaft. The protruding section 706 helps the snare loop 704 collapse from the expanded state to the delivery state when the opposite sides 708 of the loop are pressed toward each other. In one specific implementation, the loop 704 can be constructed from an 8-mil shape-set Nitinol wire. The loop 704 can alternatively be constructed from gold plated tungsten, or other suitable materials that allow flexibility, shape memory, and/or contrast under fluoroscopy.

FIG. 24 shows an alternate embodiment of a snare catheter 750 comprising an elongated shaft 752 and a snare loop 754 extending from the distal end of the shaft 752. The snare loop 754 can be shape-set such that it defines a distal protruding portion 756 and a recessed portion 758. In the expanded state of the loop (shown in FIG. 24), the recessed portion 758 wraps or extends partially around an imaginary line extending along the central longitudinal axis of the shaft 752. The recessed portion 758 can promote tension member capturing inside the body. Shapes for the snare catheters 700, 750 are not limited to those discussed above and shown in the figures. Other shapes for the snare loops, such as multiple loops, baskets, and hexagonal or asymmetrical loops, can be used.

Feeding a flexible tension member through a relatively long catheter can be difficult. Because the tension member is not rigid, advancing it through a catheter lumen can cause kinking at the insertion point, typically a leur fitting, and prevent deployment at the other end of the catheter. To prevent kinking, the tension member 402 can be affixed to one end of a small diameter wire. The wire, which can have a higher column strength than the tension member 402, can be used to pull the tension member distally through the steerable catheter 416. The wire can be, for example, a Nitinol wire having a diameter approximately the same as the diameter of the tension member.

In certain embodiments, the distal end of the wire can be advanced through the deployment catheter 500 (which extends through the steerable catheter 416) and captured by the snare catheter 700 inside the heart. The distal end of the wire can be retrieved by the snare catheter 700 and pulled into the steerable catheter 416 via the distal side opening 434. The wire, along with the tension member 402, can be pulled proximally through the lumen 452 of the steerable catheter 416 until the distal end of the tension member 402 exits the steerable catheter via the opening in the y-connector 424. Alternatively, a short length tension member can be affixed to the distal end of the wire to aid in capturing by the snare catheter 700.

In lieu of or in addition to the use of a thin wire to advance a tension member 402 through a catheter lumen, a tension member-feeding device 850 (FIG. 25) can be used to advance the tension member 402 through a catheter lumen. As shown in FIG. 25, the feeding device 850 in the illustrated embodiment comprises an inner stability tube 852 and an outer feeding tube 854, which can translate telescopingly along the inner stability tube 852 in the directions of double-headed arrow 856. In use, the distal end of the inner stability tube 852 is coupled to the proximal end of a catheter shaft 860. In the illustrated embodiment, for example, the inner stability tube 852 can be connected to a luer fitting 858 disposed on the proximal end of the catheter shaft 860. Alternatively, the distal end of the inner stability tube 852 can be removably affixed to the luer fitting 858 with a tuohy borst adapter or can be connected directly to the proximal end of the catheter shaft 860.

The inner diameter of the outer feeding tube 854 can be slightly larger than the outer diameter of the inner stability tube 852. The inner diameter of the stability tube 852 is preferably slightly larger than the outer diameter of the tension member 402.

In use, the outer feeding tube 854 can be placed around inner stability tube 852 and a tension member 402 can be fed into the inner stability tube 852 and into the catheter shaft 860. The feeding tube 854 is positioned such that a distal portion 862 surrounds the inner stability tube 852 and a proximal portion 864 surrounds a portion of the tension member 402, as depicted in FIG. 25. The proximal portion 864 can be pinched, for example, using fingers, a hemostat, or other suitable tool, such that the proximal portion is compressed against and engages the tension member 402. The feeding tube 854 is then advanced distally over the inner tube 852, thereby pushing the tension member 402 further into the catheter shaft 860. After advancing the tension member 402, the pinching force on the outer feeding tube 854 can be released and the feeding tube is retracted to the distal position to repeat the process of engaging and advancing the tension member 402 through the catheter shaft 860.

In one specific implementation, the feeding device 850 can be connected to the deployment catheter 500 and used to advance a tension member 402 through the lumen of the deployment catheter shaft 502 into the heart.

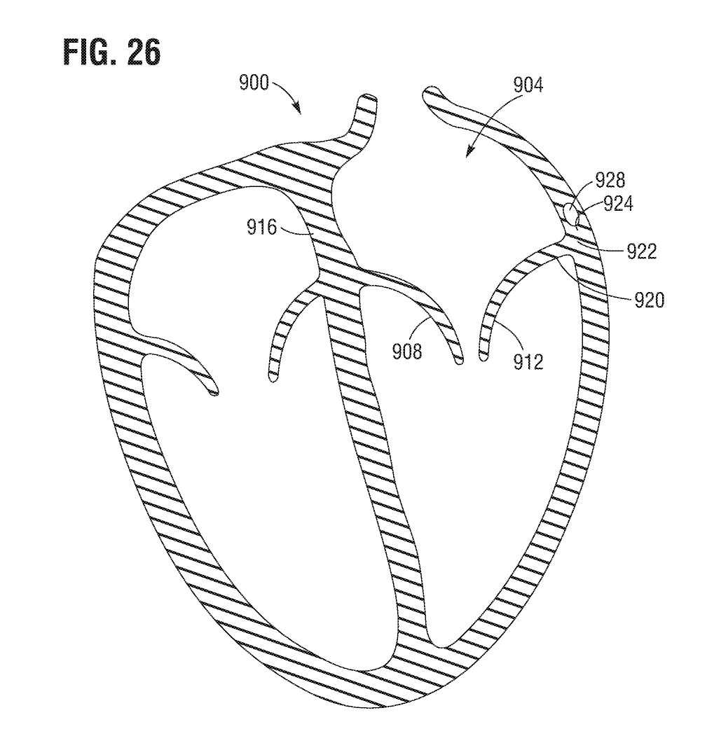

Although the methods disclosed above with reference to FIGS. 1-7 and 10-16 have been described as anchoring a tension member on or beneath the posterior mitral valve leaflet, in other embodiments, the tension member can be anchored at another location on, or proximate to, the posterior mitral valve leaflet. FIG. 26 illustrates a cross section of a heart 900, including the left atrium 904, the anterior mitral valve leaflet 908, and the posterior mitral valve leaflet 912. As described above, a proximal end of a tension member can be anchored on or proximate the interatrial septum 916. In some implementations, the distal end of the tension member can be anchored at a location 920 on or proximate to the base of the posterior mitral valve leaflet 912. For example, an anchor device can be placed underneath the posterior mitral valve leaflet 912, as described above. In other implementations, the tension member can be secured at a location 922 on or proximate the posterior annulus, or at a location 924 superior to the posterior annulus and inferior to the coronary sinus 928.

Additionally, although certain methods described above secure the distal end of the tension member using an anchor member deployed beneath the posterior mitral valve leaflet 912, the tension member can be secured in another manner, as well as at another location. For example, the tension member can be secured using an anchor disposed in or on the heart wall or leaflet tissue on or proximate one of the anchoring locations 920, 922, 924. In other examples, the tension member can be secured using an anchor disposed on the exterior surface of the heart 900 proximate one of the anchoring locations 920, 922, 924.

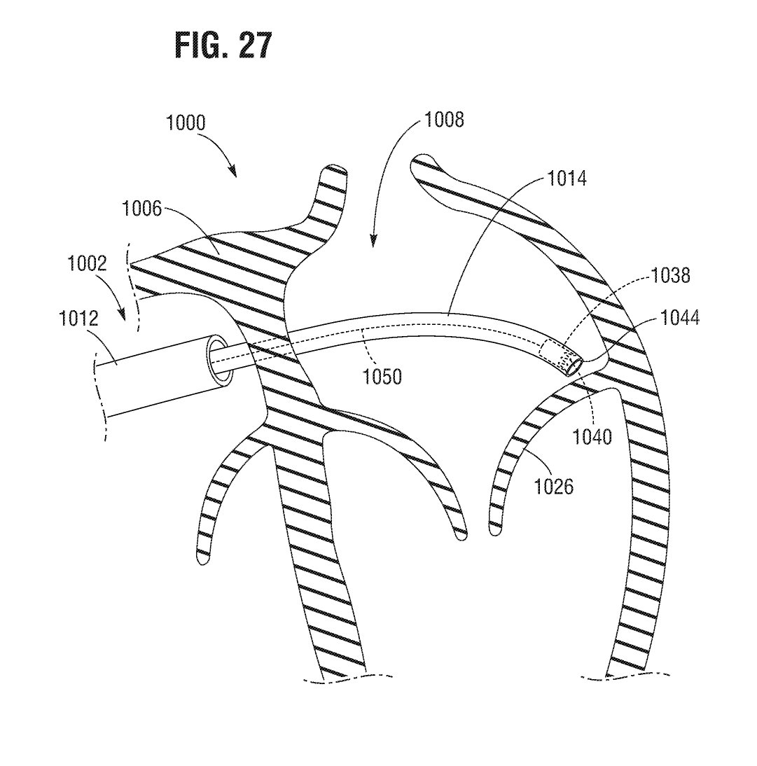

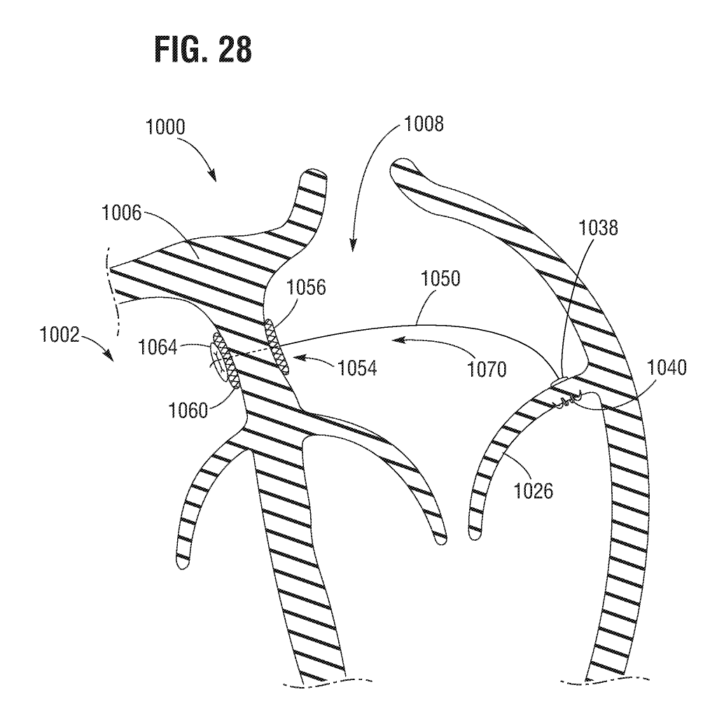

FIGS. 27 and 28 illustrate an exemplary method for delivering an anchor device transseptally to a heart 1000 to a location on or proximate to the posterior mitral valve leaflet 1026, such as from the right atrium 1002, through the interatrial septum 1006, and into the left atrium 1008. The method can include inserting a delivery catheter 1012 into the right atrium 1002. The delivery catheter 1012 can be at least substantially similar to the delivery catheter 112 of FIG. 1. A deployment catheter 1014 can extend from the delivery catheter 1012, and can be inserted into the left atrium 1008 through the interatrial septum 1006. In particular examples, the deployment catheter 1014 can be inserted through the interatrial septum 1006 on, or in an area in proximity to, the fossa ovalis.

The deployment catheter 1014 can be positioned proximate to the posterior mitral valve leaflet 1026. An anchor member 1038 can be disposed within a lumen of the deployment catheter 1014. The anchor member 1038 can include one or more gripping elements 1040, such as barbs, extending axially in a distal direction from the anchor member 1038. Although four gripping elements 1040 are shown, the anchor device 1038 can include a different number of gripping elements. The anchor device 1038 can be constructed from a shape memory alloy (such as Nitinol or another nickel-titanium alloy). The gripping elements 1040 can be heat-set such that the gripping elements extend axially when disposed within the lumen of deployment catheter 1014, and assume their heat-set shape when the anchor member 1038 is extended from a distal opening 1044 of the deployment catheter. A tension member 1050 can be coupled to the anchor member 1038 and extend proximally through the lumen of the deployment catheter 1014.

With reference to FIG. 28, when the deployment catheter 1014 has been positioned at a desired location, such as one of the locations 920, 922, 924 of FIG. 26, the anchor member 1038 can be advanced from the opening 1044 of the deployment catheter 1014. The gripping elements 1040 of the anchor member 1038 can penetrate into, and in some cases through, heart or leaflet tissue at the anchoring location. As the gripping elements 1040 are extended from the opening 1044 of the deployment catheter 1014, they can assume their heat-set shape, such as bending radially outwardly, proximally, and, optionally, radially inwardly, such as to form a hook-like shape. As the gripping elements 1040 assume their bent configuration within or through the heart or leaflet tissue, they can secure the anchor member 1038 at the anchoring location, thus securing the distal end of the tension member 1050.

With the anchor member 1038 secured by the gripping elements 1040, a proximal end of the tension member 1050 can be secured in a similar manner as described in conjunction with FIGS. 4-6. That is, a left atrial portion or anchor 1056 of a closure member 1054 can be deployed in the left atrium 1008, such as using the deployment catheter 1014. The deployment catheter 1014 can be withdrawn into the right atrium 1002, and a right atrial portion or anchor 1060 of the closure member 1054 can be deployed in the right atrium. A fastener 1064 can be advanced over the tension member 1050, placed against the right atrial portion 1060 of the closure member 1054, and secured to the tension member 1050. A desired degree of tension can be applied to the tension member 1050 in order to provide a desired remodeling force 1070 to the heart 1000.

The technologies from any example can be combined with the technologies described in any one or more of the other examples. In view of the many possible embodiments to which the principles of the disclosed technology may be applied, it should be recognized that the illustrated embodiments are only preferred examples and should not be taken as limiting the scope of the disclosed technology. Rather, the scope of the invention is defined by the following claims. We therefore claim as our invention all that comes within the scope and spirit of these claims.

What is claimed is:

1. An assembly, comprising:
an elongate delivery catheter comprising at least one lumen;
an elongate flexible tension member having first and second ends, the tension member being deployable from the catheter;
a closure member configured to be implanted in the interatrial septum of a patient's heart; and
a deployable fastener configured to be secured on the tension member adjacent to the closure member;
wherein the tension member and the anchor member cooperate to apply a remodeling force to the posterior mitral valve leaflet, improving coaptation with an anterior mitral valve leaflet.

2. The assembly of claim 1, further comprising a deployment catheter disposed within a lumen of the delivery catheter, the tension member disposed within a lumen of the deployment catheter.

3. The assembly of claim 1, wherein a portion of the tension member is formed as a loop.

4. The assembly of claim 1, further comprising an anchor device coupled to the tension member.

5. The assembly of claim 4, wherein the anchor device comprises one or more gripping elements extending axially in a distal direction, the gripping elements configured to bend radially outwardly and proximally when deployed inside the patient's heart.

6. The assembly of claim 4, wherein the anchor device is configured to expand from a delivery configuration to a deployed configuration when deployed inside the patient's heart.

7. The assembly of claim 1, wherein the closure member comprises a first portion configured to be deployed in the left atrium of the patient's heart and a second portion configured to be deployed in the right atrium of the patient's heart and the tension member extends between the first and second portions.

8. The assembly of claim 1, wherein the tension member comprises a suture.

9. The assembly of claim 1, further comprising a snare member configured to retrieve the first end of the tension member within a patient's heart and retract the first end of the tension member through the septum;

10. A method for treating a mitral valve of a heart, the mitral valve having an anterior leaflet and a posterior leaflet, the method comprising:
penetrating the posterior mitral valve leaflet;
passing a tension member through the posterior mitral valve leaflet;
securing a portion of the tension member beneath the posterior mitral valve leaflet;
coupling the tension member to a closure member implanted in the interatrial septum of the heart; and
applying a remodeling force to the native mitral valve via the tension member, the remodeling force drawing the posterior leaflet toward the anterior leaflet to promote coaptation of the leaflets.

11. The method of claim 10, wherein securing the tension member comprises forming a loop of the tension member through the posterior mitral valve leaflet.

12. The method of claim 10, wherein securing the tension member comprises deploying an anchor device connected to the tension member beneath the posterior mitral valve leaflet.

13. The method of claim 12, wherein deploying the anchor device comprises causing the anchor device to expand from a delivery configuration to a deployed configuration.

14. The method of claim 10, wherein coupling the tension member to a closure member comprises securing a fastener to the tension member proximate the closure member.

15. The method of claim 10, further comprising advancing a catheter through the interatrial septum of the heart and deploying the tension member from the catheter.

16. A method for treating a mitral valve of a heart, the mitral valve having an anterior leaflet and a posterior leaflet, the method comprising:
advancing an elongate delivery catheter into the heart;
advancing a deployment catheter through the interatrial septum of the heart to a location proximate the posterior mitral valve leaflet;
deploying a tension member from the deployment catheter;
securing a portion of the tension member at a location on or proximate to the posterior mitral valve leaflet;
applying tension to the tension member; and
securing the tension member proximate the interatrial septum;
wherein the tension member applies a remodeling force to the posterior mitral valve leaflet to promote coaptation of the mitral valve leaflets.

17. The method of claim 16, wherein advancing the deployment catheter comprising advancing the deployment catheter through the delivery catheter.

18. The method of claim 16, wherein securing the tension member comprises forming a loop of the tension member passing through the posterior leaflet and retrieving an end portion of the tension member comprises retrieving both ends of the tension member with the snare catheter.

19. The method of claim 16, wherein securing the tension member comprises deploying an anchor device coupled to the tension member at the location on or proximate to the posterior leaflet.

20. The method of claim 16, wherein the tension member is secured beneath the inferior surface of the posterior mitral valve leaflet.
